(19) United States
(12) Patent Application Publication
(10) Pub. No.: US 2004/0121455 A1
(43) Pub. Date: Jun. 24, 2004

(54) Bioreactor for manipulating biofluids at a low flow rate in a ceramic microfluidic system and method of fabrication

(76) Inventors: Rajnish Gopal Changrani, Champaign, IL (US); Frederic Zenhausern, Fountain Hills, AZ (US); Jeremy W. Burdon, Scottsdale, AZ (US); Daniel J. Sadler, Gilbert, AZ (US)

Correspondence Address: MOTOROLA, INC., CORPORATE LAW DEPARTMENT - #56-238, 3102 NORTH 56TH STREET, PHOENIX, AZ, 85018, US

(21) Appl. No.: 10/322,815

(22) Filed: Dec. 18, 2002

(51) Int. Cl. C12M 1/00

(52) U.S. Cl. 435/294.1

(57) ABSTRACT

A monolithic ceramic bioreactor for manipulating biofluids at a low flow having formed therein at least one fluid passageway, at least one electromagnetic pathway defined by a high μ magnetic material and an electrically conducting microcoil, a first and second electrode, and a biofluid comprising at least one chemical specie within a buffer solution for preserving the activity of an enzyme contained within a bioassay for genetic reaction. The device is characterized as generating a magnetic field and an electric field, perpendicular to the magnetic field, when under the application of a first and second current. In combination the magnetic field and the electric field characterized as generating a Lorentz force. The biofluid includes sufficient conductivity for fluid motion when under the influence of the Lorentz force generated within the monolithic structure, thereby providing for the manipulating of the biofluid through the monolithic structure.

### FIELD OF INVENTION

The present invention pertains to a bioreactor, and more particularly to a bioreactor for fluid manipulation at a low flow rate in a multi-layer ceramic technology, and a method of fabricating the bioreactor.

### BACKGROUND OF THE INVENTION

Laminated ceramic components containing miniature channels and other features, also referred to as microfluidic systems, which utilize low pressure lamination ceramic technology, are currently being developed for use in microfluidic management systems. Of interest is the development of microsystems based on this multilayer ceramic platform in which highly integrated functionality is key. Monolithic structures formed of these laminated ceramic components provide for three-dimensional structures that are inert and stable to chemical reactions and capable of tolerating high temperatures. In addition these structures provide for miniaturization of component parts, with a high degree of electronic circuitry or components embedded or integrated into such a ceramic structure for system control and functionality. Potential applications for these integrated devices include fluidic management in micro-channel devices for life sciences and portable fuels cell applications. One application in particular is the use of ceramic materials to form microchannels and cavities within a ceramic structure and the pumping, or movement of fluid through these microchannels and cavities. Currently, bioreactors of this type, typically referred to as magnetohydrodynamic micropumps, are provided for use but require positioning on an exterior of a ceramic package, thereby utilizing valuable circuitry real estate.

Magnetohydrodynamic (MHD) magnetohydrodynamic micropumps, or bioreactors, have been developed for use in conjunction with many devices. Although MHD pumping is not a new technology, it has recently been investigated as one means of fluid manipulation for biological microfluidic systems. One especially attractive feature of MHD is that no moving parts are required to generate the pumping action. Rather, the operation principle is based on the generation of Lorenz forces on ions within an electrolytic solution by means of perpendicular electric and magnetic fields. These Lorenz forces propel the ions through a channel, thus creating a net flow. Bi-directional flow is achievable with MHD pumping by simply adjusting the phase angle between the driving electric and magnetic fields.

Many of these pump devices are cumbersome and complex, consisting of several discrete components connected together with plumbing and hardware to produce the pump device. Of greatest concern is the inclusion of an electromagnet for control of the pump and the necessity up until this point to have it located exterior the device. Accordingly, these types of pumps have not been found suitable for portable ceramic technology applications, or in other applications requiring minimal size and weight. In an attempt to miniaturize and integrate components for use in current microsystem technologies, there exists a need for a magnetohydrodynamic micropump, such as a magnetohydrodynamic micropump that provides for integration with a ceramic laminate structure. By integrating the magnetohydrodynamic micropump, including the integrated coils, into the ceramic laminate materials, the surface area of the ceramic device can be utilized for other components, such as electrical interconnects or the like. To date, no magnetohydrodynamic micropump has been developed utilizing ceramic monolithic structures in which the miniaturization and complete integration of the pump has been achieved.

Accordingly, it is an object of the present invention to provide for a bioreactor for manipulating biofluids at a low flow rate in a ceramic microfluidic system and a method of forming the bioreactor that is fully integrated into a multi-layer ceramic structure, thereby providing for microfluidic management of a device.

It is yet another object of the present invention to provide for an integrated bioreactor, also referred to as a magnetohydrodynamic pump, and a method of forming the device for the purpose of manipulating fluids through a multilayer ceramic structure.

It is another object of the present invention to provide for an integrated bioreactor and a method of forming the device that includes no moving parts, and provides for a continuous, bi-directional flow of a solution in response to a low driving voltage.

It is another object of the present invention to provide for an integrated bioreactor and a method of forming the device that is miniaturized for use in conjunction with microsystem technologies.

It is yet another object of the present invention to provide for a method of manipulating fluids through a microsystem using an integrated magnetohydrodynamic pump.

### SUMMARY OF THE INVENTION

The above problems and others are at least partially solved and the above purposes and others are realized in a bioreactor, or magnetohydrodynamic micropump, and method of fabricating the device for the purpose of manipulating fluid in a microfluidic system. The integrated device is formed within a plurality of layers, in which the device is completely integrated into the layered structure. The system for manipulating fluid through a microfluidic system includes the magnetohydrodynamic pump, a reservoir, a microchannel, interconnects for fluid flow, and circuitry for generating electric and magnetic fields.

### BRIEF DESCRIPTION OF THE DRAWINGS

The novel features believed characteristic of the invention are set forth in the claims. The invention itself, however, as well as other features and advantages thereof will be best understood by reference to detailed descriptions which follow, when read in conjunction with the accompanying drawings, wherein:

FIG. 1 is a simplified isometric view of an integrated magnetohydrodynamic micropump according to the present invention;

FIG. 2 is a simplified sectional view of a magnetohydrodynamic micropump taken through line 2-2 of FIG. 1 according to the present invention;

FIG. 3 is a simplified isometric view of a magnetohydrodynamic micropump according to the present invention;

FIGS. 4-9 are simplified isometric and sectional views of a first and second embodiment of the microcoil portion of the magnetohydrodynamic micropump of the present invention; and

FIG. 10 is a simplified diagram of a fluidic system including an integrated magnetohydrodynamic micropump according to the present invention.

### DESCRIPTION OF THE PREFERRED EMBODIMENTS

Disclosed is the fabrication of a bioreactor, or magnetohydrodynamic pump, by means of a new ceramic MEMS (CMEMS) platform in which devices are built from multiple layers of green-sheet ceramics or other multi-layer system such as printed circuit boards, or the like. In this platform, features are formed by mechanical punching, screen-printing, and via-filling of multiple layers which are then assembled and, in the case of ceramics, fired at a high temperature. The major advantage to this technology is that unlike glass, plastic and silicon technologies, the multi-layer MEMS platform is a truly three-dimensional technology. This enables the building of complex integrated systems of micromachined flow channels along with electrodes, electrical interconnects, and active components such as pumps, valves, and sensors within a single platform.

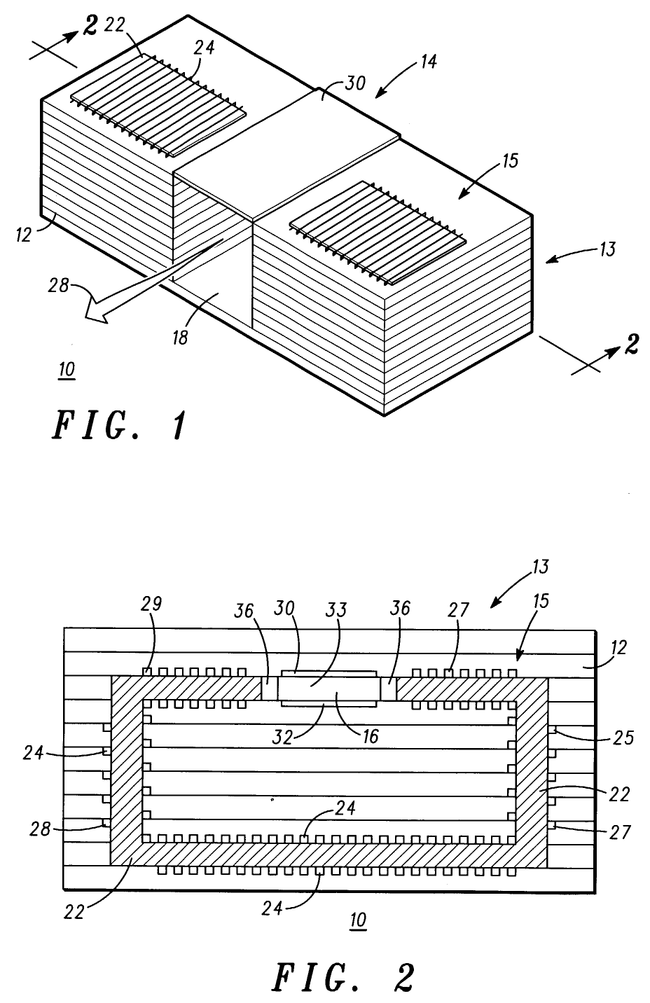

Turning now to the drawings, and in particular FIGS. 1-3, illustrated in simplified isometric and sectional views is a monolithic structure defining therein a bioreactor according to the present invention. Referring more specifically to FIGS. 1 and 2 illustrated in simplified orthogonal view, and sectional view, respectively, is a bioreactor 10, comprised of a plurality of ceramic layers 12, that once fired, sinter into a single monolithic device or monolithic structure 13. Device 10 has integrated and defined therein at least one fluid passageway 16 and at least one electromagnetic pathway 15. More specifically, device 10 has defined therein a fluid inlet 14, the fluid passageway, or channel, 16, a fluid outlet 18, and an integrated electromagnetic pathway 15 which acts as an electromagnet. Electromagnetic pathway 15 is defined by a high μ magnetic material 22, and an electrically conducting microcoil, or coil conductor, 24, formed of a conductive material that is screenprinted on ceramic layers 12 and/or formed within a plurality of interconnecting electrically conductive vias that define microcoil 24. Microcoil 24 in combination with magnetic material 22 provides for generation within the monolithic structure 13 a sustained magnetomotive force, and thus a magnetic field (B) by containing and guiding a magnetic flux generated by the electromagnetic pathway 15 when under the influence of a first current. The magnetic field (B) is indicated by directional arrow 26. A plurality of electrodes, including a first electrode 30 and a second electrode 32, spaced on opposed sides of channel 16, provide for an electric field (E), indicated by directional arrow 34 to be exerted perpendicular to the magnetic field (B), when under the influence of a second current. In combination, the magnetic field (B) and the electric field (E) generate a Lorentz force, thereby providing for the manipulation of a biofluid 33 through the fluid passageway defined in the multilayer, monolithic structure 10. Biofluid 33 is comprised of at least one chemical specie with a buffer solution for preserving the activity of an enzyme contained within a bioassay for genetic reaction, wherein the biofluid includes sufficient conductivity for fluid flow when under the influence of the Lorentz force generated within structure 10. Fluid inlet 14 provides for the intake of biofluid 33 into monolithic device 10. Microchannel 16 through which fluid flow (F) is sought, indicated by directional arrow 28, is provided in microfluidic communication with fluid inlet 14 and a fluid outlet 18.

As indicated in FIG. 2, there is provided a gap 36 defined between electromagnetic pathway 15 and fluid flow channel 16. Gap 36 in this particular embodiment is formed of a ceramic material and provides for insulation of electromagnetic pathway 15 from electrodes 30 and 32 and fluid flow channel 16. In a preferred embodiment gap 36 has a dimension of approximately 100 microns, but it should be understood that a minimum gap 36 dimension is preferred to allow for increased control of generated magnetic field 26.

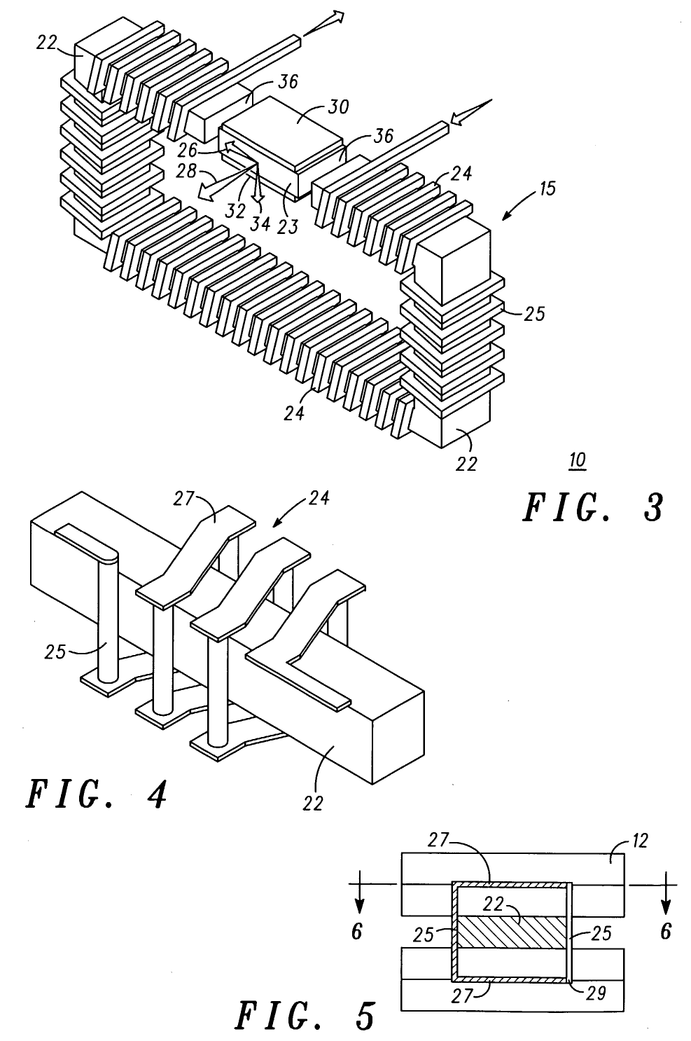

Referring now to FIG. 3, as illustrated in simplified isometric view, electromagnetic pathway 15 is formed in this preferred embodiment as a typical C-shaped electromagnet which allows for increased control of magnetic field 26 exerted by electromagnetic pathway 15. As indicated, magnetic field 26 is in a general direction as indicated, and electric field 34 is in a generally perpendicular direction to magnetic field 26, which provides for the flow 28 of biofluid 33 in fluid flow channel 16 in a direction perpendicular to magnetic field 26 and electric field 34, as per the created Lorentz force.

During operation of bioreactor 10, the generation of a Lorentz force due to the perpendicular alignment of the magnetic field 26 and electric field 34, provides for the manipulation, or flow, of biofluid 33, contained in fluid flow channel 16, in a direction perpendicular to magnetic field 26 and electric field 34.

The previously described elements of device 10 are formed in the plurality of ceramic layers 12 by means of mechanical punching, screen-printing, and via-filling of multiple layers 12 which are then assembled and fired at a high temperature, approximately 870° C. when formed of a low temperature cofired ceramic (LTCC) material. It is anticipated by this disclosure that in addition to a LTCC material forming layers 12, that alternatively a high temperature cofired ceramic (HTCC) material, or any other suitable material capable of forming a layered device can be utilized, such as a printed circuit board (PCB). As disclosed, in a preferred embodiment, ceramic layers 12 are comprised of a composite of any powdered ceramic material dispersed in an organic binder, normally a thermal plastic. This organic binder provides the starting “green sheet” material which can be handled much like a sheet of paper. LTCC materials are designed to sinter between 800° and 900° C. Use of this type of material provides for silver metallization to be used in forming via interconnect metallization and surface conductors (discussed presently).

Referring again to FIG. 2, illustrated in simplified sectional view taken along line 2-2 of FIG. 1 is bioreactor 10 of the present invention. As illustrated, monolithic structure 13 includes the plurality of ceramic layers 12 into which is integrated the bioreactor, or magnetohydrodynamic pump, 10 of the present invention. As previously described, device 10 includes electromagnetic pathway 15, including high μ material 22 and microcoils 24, and a first electrode 30 and a second electrode 32 positioned on opposed sides of a channel 16. Materials that are suitable for use as magnetic material 22 are nickel-zinc based ferrite materials, such as (Ni,Zn,Cu)O, (Ni,Zn,Fe)O, (Ni,Zn,Co)O and mixtures of these materials, e.g., (Ni,Zn,Cu,Fe)O, etc. and magnesium-zinc based ferrite materials, such as (Mn,Zn,Cu)O, (Mn, Zn, Fe)O, (Mn,Zn, Co)O and correspondent mixtures, etc.

As previously stated, device 10 is integrated into layers 12. In doing so, microcoil 24 is defined by plurality of layers 12 through screen-printing and/or formed a plurality of interconnecting vias 25. As previously stated and as illustrated in FIG. 2, microcoil 24 includes a plurality of interconnecting vias 25 that are formed by mechanically punching or laser drilling into each individual ceramic layer 12 to define a single connective via into which a conductive material 29 is positioned and/or by screenprinting traces 27 of a conductive material 29 on a surface of an individual layer 12. It should additionally be understood that emerging technologies can be utilized to form these internal structures into ceramic layers 12, such as through the use of fugitive materials thereby forming the internal cavities and channels.

Referring now to FIGS. 4-9, illustrated are a plurality of embodiments for forming electromagnetic pathway 15, and in particular the integration of microcoil 24. Illustrated in FIGS. 4-6 is a first embodiment for forming microcoil 24 in which microcoil 24 is formed parallel with ceramic layers 12. More specifically as illustrated in FIGS. 4-6, a plurality of conductive traces 27 and vias 25 having a conductive material 29 formed therein are illustrated in which traces 27 and vias 25 are interconnected through layers 12, thereby forming microcoil structure 24. As illustrated, high-μ magnetic material 22 is formed within the structure defined by microcoil 24, or more specifically is wrapped by microcoil 24. Microcoil 24 as indicated in this particular embodiment, is formed running parallel with layers 12.

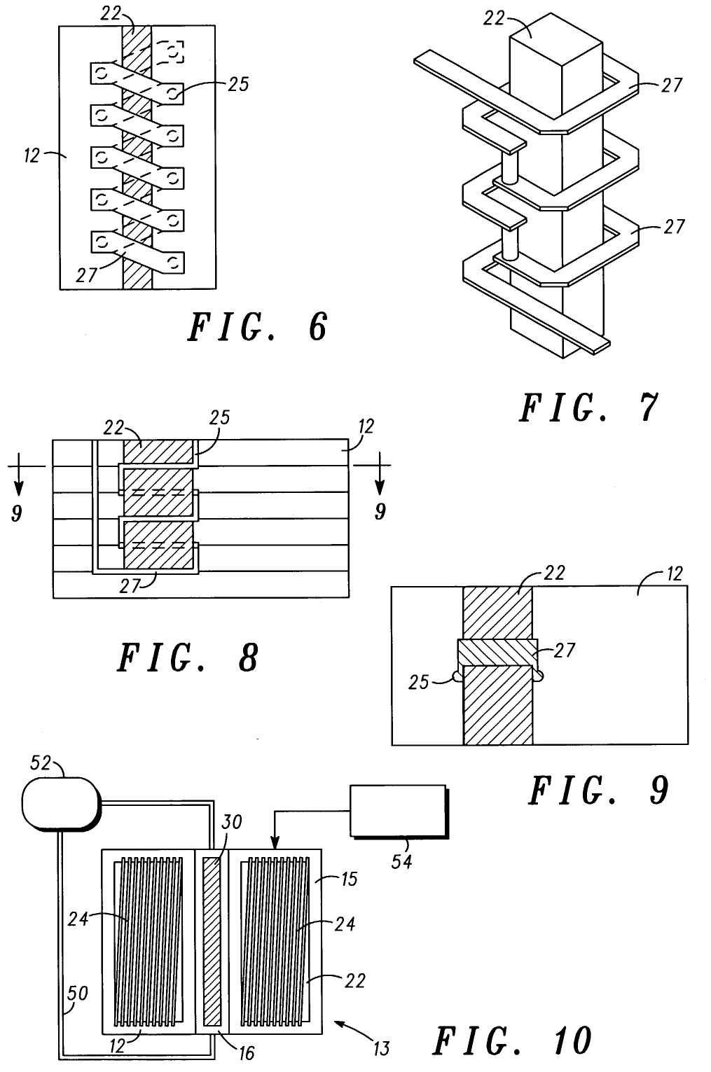

Referring now to FIGS. 7-9, illustrated is a second embodiment for forming microcoil 24 in which microcoil 24 is formed vertical to ceramic layers 12. More specifically, as illustrated in FIGS. 7-9, plurality of conductive traces 27 and vias 25 having a conductive material 29 formed therein are illustrated in which traces 27 and vias 25 are interconnected through layers 12, thereby forming microcoil structure 24. As illustrated, high-mu magnetic material 22 is formed within the structure defined by microcoil 24. Microcoil 24 as indicated, is formed running perpendicular to layers 12. It should be understood that a combination of the illustrated embodiments is typically utilized to form microcoil 24 having components formed parallel with layers 12 and perpendicular to layers 12.

During fabrication of electromagnetic pathway 15, a conductive fillable paste, or the like is utilized to form microcoil 24 within interconnecting vias 25. Standard screen-printing techniques are utilized to form traces 27 which interconnect with the conductive material formed in vias 25, to form microcoil 24. In the alternative, it is anticipated by this disclosure that traces 27 can alternatively be microchannels that are formed in the surface of ceramic layers 12 having a conductive material formed therein and interconnecting with conductive material formed in vias 25. As illustrated, traces 27 and vias 25 are formed in ceramic layers 12 to define conductive microcoil 24 within ceramic layers 12, and more particularly integrated within monolithic structure 13.

Once the component parts of bioreactor 10 are formed within layers 12, to complete monolithic structure 13, each layer is inspected and the plurality of ceramic layers 12 are laminated together to form monolithic structure 13. A low pressure lamination process is used on the stack of processed ceramic layers without collapsing the channels, vias, and other defining components formed in ceramic layers 12. This laminating process forms a compact, monolithic structure. Next, the monolithic structure is fired, or sintered, at a temperature that is less than the temperature at which the conductive materials and high-mu material, integrated therein, become unstable. More specifically, sintering at a temperature of approximately 850-900° C. is performed, whereby the organic materials are volatilized and the monolith becomes a three-dimensional functional ceramic package. It should be understood that as previously described it is anticipated by this disclosure that either a fillable high-μ paste material can be utilized to form the high-μ portion of electromagnetic pathway 15, or that a pre-formed high-μ material that can be positioned within a via formed in layers 12 and subsequently cofired with the ceramic layers 12 can be used.

Referring now to FIG. 10, illustrated in simplified diagrammatical form is a fluidic system 50, including a bioreactor 10 according to the present invention. More particularly, illustrated is a device 10, having in fluidic communication therewith, interconnect 50 for fluid flow, and reservoir 52. Device 10 provides for the pumping therethrough interconnects 50 of a fluid. Circuitry 54 provides for the delivery of a first current to electrodes 30 and 32, thereby providing for the generation of electric field 34, and the delivery of a second current to microcoil 24, thereby providing for the generation of magnetic field 36, perpendicular to electric field 34. As ions move through a fluidic solution in channel 16, fluid particles are dragged along by viscous drag forces, due to the generated Lorentz force, thus resulting in a net flow.

Accordingly, described is a bioreactor, also referred to as a magnetohydrodynamic micropump and system, that is integrated into a plurality of ceramic layers, thereby forming a monolithic ceramic package. The monolithic ceramic package provides for a bi-directional, continuous flow (non-pulsating) of fluids therethrough under the influence of a low driving voltage, without the need for moving parts. The device is formed to provide for the generation of an electric field and a magnetic field, perpendicular to the electric field, thereby creating a Lorentz force and providing for the flow of fluid through an integrated channel. The device provides for integration within the monolithic structure, all component parts which enables the building of complex integrated systems of micromachined flow channels along with electrodes, electrical interconnects, and active components such as pumps, valves, and sensors within a single platform. The three-dimensionality of multi-layer architecture, provides for the imbedding of the source of the magnetic field within the microdevice, and eliminates the need for an external electromagnet in a device having planar geometry.

While we have shown and described specific embodiments of the present invention, further modifications and improvements will occur to those skilled in the art. We desire it to be understood, therefore, that this invention is not limited to the particular forms shown and we intend in the appended claims to cover all modifications that do not depart from the spirit and scope of this invention.

What is claimed is:

1. A bioreactor for manipulating biofluids at a low flow rate comprising:
a monolithic structure having at least one fluid passageway and at least one electromagnetic pathway defined by a high μ magnetic material and an electrically conducting microcoil, the electrically conducting microcoil including a conductive material therein, and formed to wrap around a high μ magnetic material characterized as generating within the monolithic structure a sustained magnetomotive force, and thus a magnetic field, by containing and guiding a magnetic flux generated by the electromagnetic pathway;
a first and second electrode defined therein the multilayered, monolithic structure and characterized as generating within the monolithic structure an electric field, perpendicular to the magnetic field, when under the application of a current, in combination the magnetic field and the electric field characterized as generating a Lorentz force thereby providing for the manipulating of a biofluid through the at least one fluid passageway defined in the multilayer, monolithic structure; and
a biofluid comprising at least one chemical specie within a buffer solution for preserving the activity of an enzyme contained within a bioassay for genetic reaction, wherein the biofluid includes sufficient conductivity for fluid motion when under the influence of the Lorentz force generated within the monolithic structure.

2. A bioreactor for manipulating biofluids at a low flow rate as claimed in claim 1 wherein the electrically conductive microcoil is formed by a plurality of interconnecting vias formed in the plurality of layers forming the monolithic structure, the plurality of interconnecting vias having the conductive material formed therein and connecting thereto a plurality of screenprinted traces formed on the surface of the plurality of layers, thereby defining a microcoil.

3. A bioreactor for manipulating biofluids at a low flow rate as claimed in claim 1 wherein the electrically conductive microcoil is formed by a plurality of interconnecting channels and vias formed in the plurality of layers forming the monolithic structure, the plurality of interconnecting channels and vias having a conductive material formed therein, thereby defining a microcoil.

4. A bioreactor for manipulating biofluids at a low flow rate as claimed in claim 1 wherein the monolithic structure is formed of a plurality of sintered ceramic layers, having integrated therein the at least one fluid passageway and at least one electromagnetic pathway defined by a high μ magnetic material and an electrically conducting microcoil, and the first and second electrodes.

5. A bioreactor for manipulating biofluids at a low flow rate as claimed in claim 1 wherein the monolithic structure is formed of a plurality of layers formed on a printed circuit board, having integrated therein the at least one fluid passageway, and the at least one electromagnetic pathway defined by the high μ magnetic material and the electrically conducting microcoil, and the first and second electrodes.

6. A bioreactor for manipulating biofluids at a low flow rate as claimed in claim 1 wherein the magnetic material is comprised of a high μ material that is positioned within in the plurality of layers and cofired with the plurality of ceramic layers.

7. A bioreactor for manipulating biofluids at a low flow rate as claimed in claim 6 wherein the magnetic material is a pre-formed bulk magnetic material.

8. A multilayer ceramic bioreactor for manipulating biofluids at a low flow rate comprising:
a monolithic structure formed from a plurality of layers;
an electromagnetic pathway formed within the plurality of layers, the electromagnetic pathway comprised of a high μ material and a microcoil defined by a plurality of interconnecting vias formed in the plurality of layers and having a conductive material positioned therein the interconnecting vias, the microcoil formed to wrap around the high μ material, the electromagnetic pathway characterized as generating a magnetic field within the monolithic structure when under the application of a first current;
a first electrode and second electrode defined therein the monolithic structure, characterized as generating an electric field, perpendicular to the magnetic field, when under the application of a second current;
a fluid flow channel defined therein the monolithic structure and in perpendicular alignment with the magnetic field and the electric field, whereby in combination the magnetic field and the electric field generate a Lorentz force thereby providing for the manipulating of a biofluid through the fluid flow channel and thus through the monolithic structure.

9. A multilayer ceramic bioreactor for manipulating biofluids at a low flow rate as claimed in claim 8 wherein the microcoil is defined by the plurality of interconnecting vias and a plurality of screenprinted traces formed on the surface of the plurality of layers, the plurality of interconnecting vias and the screenprinted traces thereby defining the microcoil.

10. A multilayer ceramic bioreactor for manipulating biofluids at a low flow rate as claimed in claim 8 wherein the microcoil is defined by the plurality of interconnecting vias and a plurality of channels formed in the plurality of layers forming the monolithic package, the plurality of interconnecting vias and channels thereby defining the microcoil.

11. A multilayer ceramic bioreactor for manipulating biofluids at a low flow rate as claimed in claim 8 wherein the multilayer package is formed of a plurality of sintered ceramic layers, having integrated therein the magnetic material and the microcoil, the first and second electrodes, and the channel for the flow of a fluid through the plurality of sintered ceramic layers.

12. A multilayer ceramic bioreactor for manipulating biofluids at a low flow rate as claimed in claim 8 wherein the multilayer package is formed of a plurality of layers formed on a printed circuit board, having integrated therein the magnetic material and the microcoil, the first and second electrode, and the channel for the flow of a fluid through the plurality of layers.

13. A multilayer ceramic bioreactor for manipulating biofluids at a low flow rate as claimed in claim 8 wherein the magnetic material is comprised of a high μ material that is positioned within in the plurality of layers and cofired with the plurality of ceramic layers.

14. A multilayer ceramic bioreactor for manipulating biofluids at a low flow rate as claimed in claim 8 wherein the magnetic material is a pre-formed bulk magnetic material.

15. A method for manipulating a biofluid in a bioreactor at a low flow rate including the steps of:
providing a plurality of ceramic layers;
forming into the plurality of ceramic layers an electromagnetic pathway comprised of a plurality of interconnecting vias formed in the plurality of ceramic layers, thereby defining a microcoil, the plurality of interconnecting vias formed to wrap around a high μ material;
forming into the plurality of ceramic layers a fluid flow channel having a first electrode and a second electrode formed on opposed sides of the fluid flow channel;
laminating each of the plurality of ceramic layers having the electromagnetic pathway, the fluid flow channel and the first and second electrodes formed therein, to form a ceramic monolithic package;
sintering the monolithic package to form a functional monolithic three-dimensional magnetohydrodynamic micropump device defining therein;
introducing a biofluid comprising at least one chemical specie within a buffer solution for preserving the activity of an enzyme contained within a bioassay for genetic reaction, wherein the biofluid includes sufficient conductivity for fluid motion when under the influence of the Lorentz force generated within the monolithic structure.

16. A method for manipulating a biofluid in a bioreactor at a low flow rate as claimed in claim 15 wherein the step of providing a plurality of ceramic layers includes the step of providing a plurality of green sheets comprised of a ceramic material dispersed in an organic binder.

17. A method for manipulating a biofluid in a bioreactor at a low flow rate as claimed in claim 16 wherein the step of forming into the plurality of ceramic layers a plurality of channels and interconnecting vias includes forming the channels and interconnecting vias by at least one of mechanically punching or laser drilling into each individual ceramic layer.

18. A method for manipulating a biofluid in a bioreactor at a low flow rate as claimed in claim 16 wherein the step of sintering the monolithic package to form a functional monolithic three-dimensional magnetohydrodynamic micropump device includes sintering the laminated structure at a temperature less than the temperature at which the high μ material becomes unstable.
